(19) United States
(12) Patent Application Publication
(10) Pub. No.: US 2006/0167094 A1
(43) Pub. Date: Jul. 27, 2006

(54) Satiety agent

(76) Inventors: Donald Fleming, Adelaide (AU); Christine Feinle-Bisset, Adelaide (AU)

Correspondence Address: NATH & ASSOCIATES, 112 South West Street, Alexandria, VA, 22314, US

(21) Appl. No.: 11/136,457

(22) Filed: May 25, 2005

(30) Foreign Application Priority Data
Jan. 27, 2005 (AU) 2005200306

(51) Int. Cl. A61K 31/22

(52) U.S. Cl. 514/546; 514/547

(57) ABSTRACT

A composition and method for the control of appetite, having as an active ingredient a satiety agent and a pharmaceutically acceptable delivery agent, formulated so that the release of the active ingredient is predominately in the stomach.

The invention relates to the use of satiety agent for the reduction or control of obesity in humans. In particular, the invention relates to a composition and also a method that can assist in appetite suppression and energy intake in humans.

### DESCRIPTION OF THE PRIOR ART

Obesity is an increasing problem in today's society, one that affects all ethnic, age and socioeconomic groups. It has been estimated that there are more than 250 million obese people worldwide, which represents approximately 7% of the adult population. This is clearly a global problem, one that greatly affects developed countries in which there is little food deprivation and reduced levels of physical activity.

Obesity is not merely a problem relating to physical appearance but rather one that has far reaching implications in relation to overall health and lifestyle of individuals. There is a growing body of evidence that suggests that excess body weight is directly associated with an increase in the risk of various forms of cancer, including colon, breast and kidney cancer.

Additionally, obesity is associated with type-2 diabetes, hypertension, sleep apnoea, dyslipidaemia and osteoarthritis.

The level of obesity in humans is determined by calculating an individual's body mass index or BMI and if >25 classified as overweight. If BMI is >30 this is considered obese and if >40 then this is classified as severely obese.

In the United States, for example, there has been an increase in the prevalence of the number of adults categorized as being either overweight, obese or severely obese. Notably, in the period 1988 to 1994, 23% of the American population was considered to be obese. From the period 1999 to 2000, this number had risen to 30.5%.

The number of obese individuals in Australia is also alarmingly high, with the number doubling over the last 20 years. Currently some 53% of Australian women and 64% of Australian men are categorised as being overweight. Of more concern are the figures relating to the number of Australians considered to be obese, 22% of women and 19% of men.

Perhaps even more alarming, the World Health Organisation estimates that there are approximately 17.5 million children world wide under the age of five that fall in the obese category.

This alarming trend is set to continue and the full impact on society is yet to be truly felt.

There are a number of pharmacological treatments currently available that attempt to address human obesity. Apart from a change of diet and lifestyle, such treatments include weight control agents that act directly on the central nervous system (CNS) in an attempt to suppress appetite. These include amphetamines, such as dextroamphetamine, which have the potential to cause addiction.

Sibutramine (Meridia®, Abbot Laboratories) is a catecholaminergic and serotonergic agonist that regulates the feeling of fullness (satiety) from within the brain. Side effects of sibutramine include dry mouth, headaches, insomnia, constipation, increases in heart rate and blood pressure.

Additionally, pharmacological treatments such as orlistat (Xenical®, Roche) that inhibit pancreatic and gastric lipases, thus decreasing the digestion of fat as well as absorption of fat into the bloodstream, result in unabsorbed fats being passed out in the faeces. However, this can result in considerable gastrointestinal side effects, such as loose stools and increased flatulence. Moreover, it has been shown that the inhibition of fat digestion has been associated with compensatory increase in subsequent energy intake (1).

Both sibutramine and orlistat have been approved by the US FDA.

Other treatments are classified as surgical in which an individual undergoes either gastric bypass or intestinal bypass operations. In gastric bypass a length of intestine is removed thus reducing the chance of nutrients being absorbed by the body. However, the side effects from such surgical treatment can result in liver damage or chronic diarrhea.

Gastric stapling results in the reduction in size of a patient's stomach, usually by stapling a section off resulting in early satiety and thus controlling the amount of food ultimately ingested. This particular procedure is only used in cases where the individual is considered to be severely obese.

One of the main contributions to the current state of obesity in the human population is related directly to the decrease in physical activity while maintaining a diet that is high in energy. The result is that the unused energy is stored as fat, positioned particularly around the midgut region (adipose tissue).

### OBJECT OF THE INVENTION

It is an object of the present invention to provide for use a satiety agent for the reduction or control of obesity in humans.

A further object of this invention relates to provision of a composition and also a method that can assist in suppression of appetite and energy intake in humans.

It is a further object of the invention to provide a composition that reduces energy intake in individuals.

It is a further object of the invention to provide a composition and method for the treatment of obesity that is a naturally occurring food product.

Another object of the invention is to provide a composition and method for the treatment of obesity that will be able to be used with at least minimal adverse side effects in most users.

It is yet a further object of the present invention to overcome, or at least partially ameliorate, the disadvantages and shortcomings of the prior art.

Other objects and advantages of the present invention will become apparent from the following description, taken in combination with the accompanying figures, wherein an embodiment of the present invention is disclosed.

### SUMMARY OF THE INVENTION

According to the present invention, although this should not be seen as the most narrow form, there is provided a composition for use as a satiety modifying agent including a pharmaceutically acceptable satiety agent selected from the group consisting of dodecanoic acid, glyceryl dodecanoate, glyceryl 1,3-didodecanoate and glyceryl tridodecanoate and derivatives and mixtures thereof, and a pharmaceutically acceptable delivery agent.

In preference, the composition is formulated for predominant release of the satiety agent in the stomach, which is achieved by selective use of pharmaceutically acceptable coating or encapsulating materials.

In preference, the concentration of the satiety agent is at least 80%. In preference, the concentration of the satiety agent is at least 90%.

In preference, the satiety agent is present in the amount ranging from 0.5 grams to 15 grams.

In preference, the satiety agent is present in the amount ranging from 2 grams to 10 grams.

In preference, the satiety agent is present in the amount ranging from 3 grams to 6 grams.

In preference, the satiety agent is in liquid form to facilitate dispersion upon release in the stomach.

In preference, the satiety agent is formulated to be dispersed in the stomach between 15° C. and 40° C.

In preference, the satiety agent is formulated to be to be dispersed in the stomach between 18° C and 25° C.

In a further aspect, the invention may exist in the use of a satiety agent selected from the group consisting of dodecanoic acid, glyceryl dodecanoate, glyceryl 1,3-didodecanoate and glyceryl tridodecanoate and derivatives and mixtures thereof for the manufacture of a medicament for the treatment of obesity.

In yet a further aspect of the invention, there is a method of controlling appetite including the steps of administering to a subject whose appetite is to be controlled a composition including a satiety agent selected from the group consisting of dodecanoic acid, glyceryl dodecanoate, glyceryl 1,3-didodecanoate and glyceryl tridodecanoate and derivatives and mixtures thereof, a delivery agent designed to release the satiety agent predominantly in the stomach.

In preference, the composition is ingested 5-60 min prior to the ingestion of food.

In yet a further form of the invention there is a method of increasing a feeling of satiety which includes a step of effecting by oral ingestion a composition comprising a pharmaceutically acceptable satiety agent selected from a group consisting of any one alone or any two or more in combination the group being dodecanoic acid, glyceryl dodecanoate, glyceryl 1,3-didodecanoate and glyceryl tridodecanoate and derivatives and mixtures thereof, and a pharmaceutically acceptable delivery agent which is either a capsule within which the active agent is held or a coating either coating as a whole or smaller portions the active agent or agents where the capsule or coating is such as to resist release of the active agent when orally consumed until within the stomach of a user and then allow release at least substantially of the active agent while within at least to a major extent within the stomach of a user.

In preference, the step is effected so that the ingestion is effected at least after 5 minutes and not longer than 60 minutes before commencement of expected oral partaking of food by a user.

### BRIEF DESCRIPTION OF THE DRAWINGS

FIG. 1A-D are graphical representations of results of the appetite perception scores (VAS scores) in connection with the study described below.

FIG. 2 is a graphical representation of energy intake in connection with the study described below.

FIG. 3A-B are graphical representations of antropyloroduodenal pressures in connection with the study described below.

FIG. 4A-C are graphical representations of pyloric pressures in connection with the study described below.

FIG. 5A-B are graphical representations of duodenal pressures in connection with the study described below.

FIG. 6 is a graphical representations of pressure wave sequences in connection with the study described below.

FIG. 7A-B are graphical representations of plasma CCK and GLP-1 concentrations in connection with the study described below.

### DETAILED DESCRIPTION OF THE INVENTION

Subjects

A total of 12 subjects were studied: 4 subjects in a nonrandomized pilot study and 8 subjects in the randomized study using the protocol described below. This subject number was based on “crude” power calculations derived from our previous studies The healthy, male volunteers had a mean age of 24±4 yr (range 19-47 yr) and were of normal body weight (body mass index=22.0±1.6 kg/m²). All subjects were unrestrained eaters [scoring <12 on the eating restraint part (factor 1) of the Three-Factor Eating questionnaire (2)], had no gastrointestinal disease or symptoms, and were not taking any medication known to affect gastrointestinal motility or appetite. No subject smoked or habitually consumed >20 g alcohol/day.

Preparation and Doses of Fatty Acids

Fatty acid solutions were prepared using 5.3 g of commercially available food grade saturated free fatty acids, lauric (C12:0) and decanoic (C10:0) acid (Sigma-Aldrich; Milwaukee, Wis.). C12 was dissolved in 0.89 g NaOH (Sigma-Aldrich; St. Louis, Mo.) and distilled water to a total volume of 250 ml. C12 was maintained in solution by heating it to 37° C., with a resulting pH of 8.2. C10 was also dissolved in NaOH (1.21 g), and the pH of the C10 and control (distilled water) solutions was adjusted to 8.2 by the addition of NaOH. All solutions were prepared on the morning of the study and infused at 37° C. The infusion rate was 2 ml/min, so that the total volume infused in 90 min was 180 ml, which corresponded to an energy delivery rate of 0.375 kcal/min for the fatty acid solutions (i.e., total energy administered was 33.8 kcal, or 141 kJ). We chose to infuse the fatty acid and control solutions intraduodenally to ensure uniformity of doses across subjects and study days.

A pilot study was initiated with an infusion rate of 1.5 kcal/min, comparable to a well-tolerated dose of intraduodenal triglycerides used in previous studies.

However, during the C12 infusion, the first subject tested reported severe nausea and only tolerated the infusion for 30 min. A second subject, who again reported severe nausea, tolerated the infusion for 60 min. Rates of 1 kcal/min (n=1) and, subsequently, 0.75 kcal/min (n=1) of C12 were also only tolerated for some 60 min due to severe nausea and vomiting. After these initial observations, dose of 0.375 kcal/min was chosen because C12 had been reported to empty from the human stomach at this rate and because C12 had been perfused into human duodenum at 0.36 kcal/min without adverse effects.

Protocol

Each subject was studied on three occasions, separated by 3-10 days, to evaluate, in a double-blind, randomized fashion, the effects of intraduodenal infusions of 1) C12, 2) C10, or 3) control solution for 90 min on appetite perceptions, energy intake, APD pressures, and plasma CCK and GLP-1 concentrations.

Subjects attended the laboratory at 0830 after fasting from 2200 the previous night from both solids and liquids. They were intubated with a 17-channel manometric catheter (Dentsleeve; Adelaide, South Australia, Australia) that was inserted through an anesthetized nostril and allowed to pass through the stomach and into the duodenum by peristalsis. The manometric catheter, which consisted of 16 sideholes spaced at 1.5-cm intervals, measures pressures in the antrum, pylorus, and duodenum. Six side holes (channels 1-6) were positioned in the antrum, a 4.5-cm sleeve sensor (channel 7), with two channels present on the back of the sleeve (channels 8 and 9), designed to measure pressure waves occurring over the entire pyloric region, were positioned across the pylorus, and seven channels were positioned in the duodenum (channels 10-16). An additional channel, used for intraduodenal infusions, was positioned 11.75 cm distal to the end of the sleeve sensor. The correct positioning of the catheter, so the sleeve sensor straddled the pylorus, was maintained by continuous measurement of the transmucosal potential difference (TMPD) between the most distal antral channel (channel 6; approximately −40 mV) and the most proximal duodenal channel (channel 10; ˜0 mV). For this purpose, an intravenous cannula filled with sterile saline was placed subcutaneously in the left forearm and used as a reference electrode. All manometric channels were perfused with degassed, distilled water except for the two TMPD channels, which were perfused with degassed 0.9% saline at 0.15 ml/min. An intravenous cannula was also placed into a right forearm vein for blood sampling to measure plasma CCK and GLP-1 concentrations.

Once the catheter was positioned correctly, fasting motility was monitored until the occurrence of phase III of the interdigestive migrating motor complex (MMC). Immediately after the cessation of phase III activity [at time t=−15 min], a baseline venous blood sample was taken, and a visual analog scale questionnaire (VAS) (see Measurements), assessing appetite-related sensations, was administered. At t=0 min (i.e., during phase I of the MMC), the duodenal infusion of 1) C12, 2) C10, or 3) control solution was commenced and continued for 90 min. APD pressures were monitored throughout the infusion period; blood samples were taken, and the VAS administered every 15 min from t=0 min until t=90 min. At t=90 min, the infusion was terminated, and the subject was immediately extubated and offered a cold buffet-style meal, the composition of which is provided in Table 1. The amount of food offered was in excess of what the subject was expected to consume. The subject was given 30 min (i.e., t=90 min to t=120 min) to consume the meal and instructed to eat until comfortably full. After ingestion of the meal, the subject was monitored for a further 30 min, a further VAS was completed, and blood samples were taken at t=120 and 150 min. The intravenous cannula was then removed, and the subject was allowed to leave the laboratory.

TABLE 1
Composition of the buffet meal

| | Amount | Energy | | | |
|---|---|---|---|---|---|
| | Served, | content, | Fat, | Carbohydrate, | Protein, |
| Food items | g | kJ | g | g | g |
| Wholemeal bread, | 125 | 1,304 | 3.6 | 50.0 | 12.6 |
| 4 slices | | | | | |
| White bread, | 125 | 1,295 | 2.9 | 56.4 | 11.8 |
| 4 slices″ | | | | | |
| Ham, sliced | 100 | 453 | 3.6 | 0 | 18.8 |
| Chicken, sliced | 100 | 677 | 7.0 | 0 | 24.6 |
| Cheese, sliced | 85 | 1,436 | 28.3 | 0.9 | 21.9 |
| Tomato, sliced | 100 | 56 | 0.1 | 1.9 | 1.0 |
| Lettuce | 100 | 27 | 0 | 0.4 | 0.9 |
| Cucumber, sliced | 100 | 44 | 0.1 | 1.9 | 0.5 |
| Strawberry yoghurt | 200 | 966 | 6.2 | 33.8 | 9.4 |
| Fruit salad | 140 | 343 | 0.1 | 19.3 | 0.6 |
| Chocolate custard | 150 | 662 | 5.3 | 22.7 | 4.8 |
| Apple | 170 | 359 | 0.2 | 21.3 | 0.5 |
| Banana | 190 | 680 | 0.2 | 37.8 | 3.2 |
| Orange juice, | 500 | 800 | 5.0 | 42.5 | 5.0 |
| unsweetened | | | | | |
| Iced coffee | 600 | 1,788 | 10.2 | 61.8 | 21.0 |
| Water | 600 | 0 | 0 | 0 | 0 |
| Margarine | 20 | 609 | 16.4 | 0.1 | 0.1 |
| Mayonnaise | 20 | 310 | 6.5 | 4.0 | 0.2 |
| Total | 3,425 | 11,808 | 95.7 | 354.6 | 136.9 |

Measurements

Appetite sensations and energy intake. Subjective sensations of appetite, including hunger, fullness, desire to eat, prospective consumption (“how much food do you think you could eat if you were given a meal now?”), were assessed using validated VASs (Sensations of nausea and bloating were also assessed. Each VAS evaluated a sensation on a 100-mm horizontal line, where 0 represented “sensation is not felt at all” and 100 represented “sensation is felt the greatest.” Subjects were asked to place a vertical stroke on the 100-mm line in relation to what they were feeling at that particular point in time.

Energy intake from the buffet meal [energy consumption (in kJ) and macronutrient distribution (in % and g energy)] was analyzed using commercially available software (Food works 3.0, Xyris Software; Highgate Hill, Queensland, Australia). The time taken to finish the meal (in min) was also evaluated.

APD pressures. Manometric pressures were digitized, recorded on a computer-based system (PowerMac 7100/75, Apple Computers; Cupertino, Calif.) running commercially available software (HAD, Associate Prof. G S Hebbard, Melbourne, Australia) written in LabView 3.1.1 (National Instruments), and stored for subsequent analysis. APD pressures were analysed for 1) number and amplitude of isolated pyloric pressure waves (IPPWs) and pressure waves (PWs) in the antrum and duodenum and 2) number and length of PW sequences (PWSs) in the duodenum using custom-written software (Gastrointestinal Motility Unit; Utrecht, The Netherlands) modified to our requirements. Basal pyloric pressure (“tone”) was also calculated for each minute by subtracting the mean basal pressure (excluding phasic pressures) recorded at the most distal antral side hole from the mean basal pressure recorded at the sleeve using custom-written software (MAD, Prof. Charles Malbert, Institut National de la Recherche Agronomique, Rennes, France). Phasic PWs in the antrum and IPPWs were defined by an amplitude ≧10 mmHg, with a minimum interval of 15 s between peaks. Phasic duodenal PWs were defined by an amplitude ≧10 mmHg, with a minimum interval of 3 s between peaks. Duodenal pressures were regarded as related between channels if their velocities between adjacent side holes were between 9 and 160 mm/s and thus defined as duodenal PWSs. Duodenal PWSs were characterized according to the distances travelled, i.e., over at least two (1.5 to <3 cm), three (3 to <4.5 cm), four (4.5 to <6 cm), five (6 to <7.5 cm), six (7.5 to <9 cm), and seven (9 to <10.5 cm) channels and expressed as total number of waves. Antral PWSs were not analyzed because these were infrequent.

Plasma CCK and GLP-1 Concentrations

Venous blood samples (10 ml) were collected in ice-chilled EDTA-treated tubes containing 400 kIU aprotinin (Trasylol, Bayer Australia) per millilitre of blood. Plasma was separated by centrifugation (3,200 rpm, 15 min, 4° C.) within 30 min of collection and stored at −70° C. until assayed.

Plasma CCK concentrations (in pmol/l) were determined after ethanol extraction using a previously described radioimmunoassay (3). A commercially available antibody (C258, Lot 105H4852, Sigma Chemical) raised in rabbits against the synthetic sulfated CCK-8 was employed. This antibody binds to all CCK peptides containing the sulfated tyrosine residue in position 7, shows a 26% cross-reactivity with unsulfated CCK-8 and <2% cross-reactivity with human gastrin, and does not bind to structurally unrelated peptides. The intra-assay coefficient of variation (CV) was 9% and the interassay CV was 27%, with a sensitivity of 2.5 pmol/l. The sensitivity of the assay relates to its reliable detection limit.

Plasma GLP-1 concentrations (in pmol/l) were measured by radioimmunoassay using an adaptation of a previously published method (4) Antibody, supplied by Prof. S. R. Bloom (Hammersmith Hospital, London, UK), did not cross-react with glucagon, gastric inhibitory peptide, or other gut or pancreatic peptides and has been demonstrated by chromatography to measure intact GLP-1₍₇₋₃₆₎ amide. It is likely that this antibody also reacts with the degraded form of GLP-1₍₉₋₃₆₎ amide. Intra-assay CV was 17% and interassay CV was 18%, with a sensitivity of 1.5 pmol/l.

Statistical Analysis

Baseline (“0”) values were calculated as the mean of values obtained at t=−15 and 0 min for VAS scores and plasma hormone concentrations and between t=−15 to 0 min for basal pyloric pressures, number, and amplitude of IPPWs, antral and duodenal PWs, and total number of duodenal PWSs. Basal pyloric pressures and the number and amplitude of IPPWs were expressed as means over 15-min segments during the 90-min infusion period. Numbers and amplitudes of antral and duodenal PWs were expressed as total number and mean values, respectively, for the 90-min infusion period. Duodenal PWSs were expressed as the total number of waves travelling over two, three, four, five, six, and seven channels during the 90-min infusion period. All data, except for plasma CCK and GLP-1 concentrations, were expressed as changes from baseline.

VAS scores, basal pyloric pressures, number and amplitude of IPPWs, and plasma hormone concentrations were analyzed by repeated-measures mixed model analysis of covariance (ANCOVA), with baseline as the covariate and time (0-90 min, at 15-min intervals) and treatment as within-subject factors. One-way ANOVA was used to assess the effect of treatment on energy intake, time taken to complete eating, total numbers and mean amplitudes of antral and duodenal PWs, and total number of duodenal PWSs. A logarithmic transformation was applied to nausea scores, antral amplitudes, and duodenal PWSs travelling 7.5 and 9 cm to better address the underlying assumptions of the models used (as these variables lacked normal distribution). If significance or near significance (0.05>P<0.07) was found, post hoc comparisons of least-squares means using Student's t-tests were performed. Raw P values for post hoc tests, not adjusted for multiple comparisons, have been reported, because the aim of the study was to describe the effect of treatment and time on various responses and not to test specific a priori hypotheses. Linear associations were assessed between 1) the desire to eat and 2) nausea with plasma CCK, plasma GLP-1, the sum of plasma CCK and GLP-1 (calculated by adding plasma CCK and GLP-1 concentrations at each time point), basal pyloric pressure, and number and amplitude of IPPWs, by calculating correlation coefficients adjusted for repeated measures. Statistical analysis was performed using SAS (SAS Institute; Cary, N.C.). Statistical significance was accepted at P<0.05, and data are presented as means±SE.

### RESULTS

The subjects, with the exception of one, tolerated all experimental conditions well. The subject, who was able to tolerate the C12 infusion for only 30 min due to severe nausea (and was dismissed at that time), completed the two other infusions successfully. All available data were included in the analyses.

Appetite Perceptions (VAS Scores)

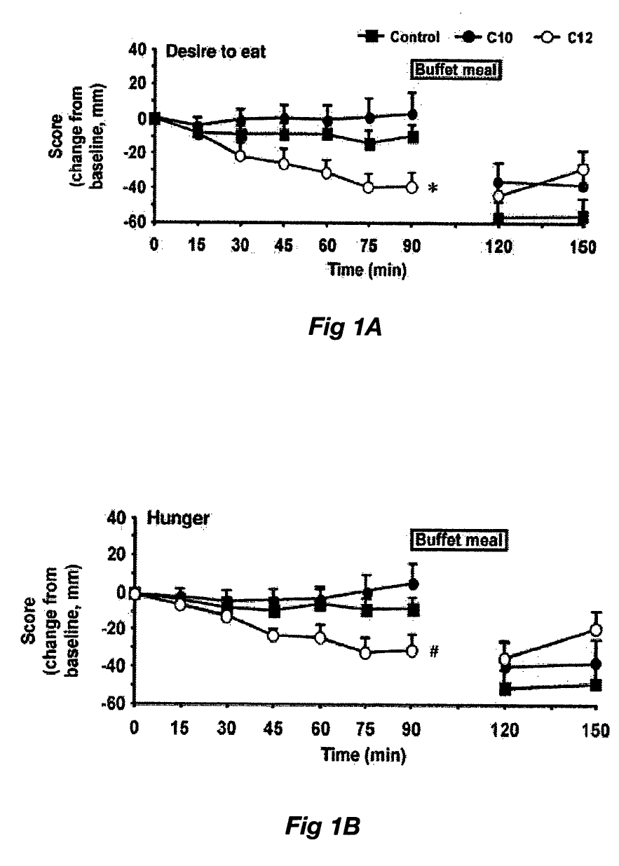

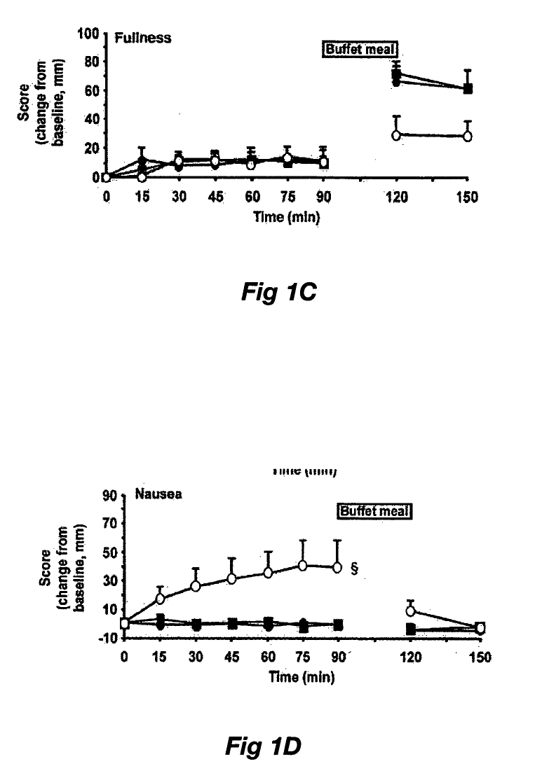

There was a significant effect of treatment on scores for the desire to eat (FIG. 1A), hunger (FIG. 1B), and prospective consumption (data not shown) (P<0.001 for all). During the infusion of C12, scores for desire to eat, hunger, and prospective consumption were lower compared with control (P<0.001 for all) and C10 (P<0.001 for all), whereas no differences were found between C10 and control [not significant (NS)]. There was a small, but significant, increase in fullness (FIG. 1C) over the infusion period with all three treatments (time effect: P=0.034), without any differences between them (treatment effect: NS).

There was a significant effect of treatment on nausea (FIG. 1D); P<0.001). Infusion of C12 increased nausea scores compared with control (P<0.001) and C10 (P<0.001). Five of the eight subjects reported nausea, whereas the other three subjects did not experience nausea. There was no difference in nausea scores between C10 and control (NS). There was a significant effect of treatment on bloating (P<0.001; data not shown). Infusion of C12 increased the scores for bloating compared with both C10 (P<0.001) and control (P<0.001). C10 also increased scores for bloating compared with control (P=0.013).

After ingestion of the meal, scores for the desire to eat (FIG. 1A), hunger (FIG. 1B), and prospective consumption (data not shown) decreased significantly (time effects: P<0.05), but there were no differences between treatments at 120 or 150 min (treatment effects: NS). Scores for fullness increased after meal ingestion (FIG. 1C; time effect: P=0.001), and at 120 and 150 min fullness was less after C12 than C10 (P=0.042) or control (P=0.012), with no difference between C10 and control (NS; treatment effect: P=0.030).

Scores for both nausea (FIG. 1D) and bloating (data not shown) diminished rapidly in the nauseated subjects after termination of the C12 infusion at 90 min; by 120 min, the mean score for nausea was close to baseline, and there were no significant differences between treatments at 120 and 150 min (NS).

Energy Intake

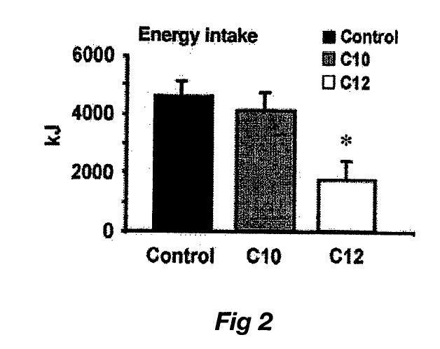

There was a significant effect of treatment on energy intake (P=0.001; FIG. 2). Infusion of C12 dramatically decreased energy intake compared with control (P<0.001) and C10 (P<0.001), whereas there was no difference between C10 and control infusion (NS). The decrease in energy intake after the C12 infusion was evident in all subjects; however, the magnitude of the reduction was greater in those subjects who reported nausea (˜3,840 kJ) compared with those that did not experience nausea (˜1,801 kJ) (because of the small subject numbers in each “subgroup,” these data were not subjected to formal statistical analysis).

There was also a significant effect of treatment on the macronutrient distribution of the food consumed (Table 2; P<0.001 for all). The infusion of C12 reduced the amount of energy (in g) ingested from fat, carbohydrate, and protein compared with control (fat: P<0.001; carbohydrate: P<0.001; protein: P<0.001) and C10 infusion (fat: P=0.001; carbohydrate: P=0.002; protein: P=0.003), whereas there was no difference between C10 and control (NS for all).

The time at which eating from the buffet meal was completed tended to be less after C12 infusion (13±3.5 min) compared with C10 (20.6±2.6 min) or control (21.8±1.8 min); however, this did not reach statistical significance (NS).

Antropyloroduodenal Pressures

Visual inspection of the pressure traces suggested that intraduodenal infusion of C12 resulted in a typical “fed” motor pattern in which antral and duodenal PWs were inhibited and pyloric pressures stimulated. A duodenal phase III episode occurred in two subjects: in one subject during the control and in the other subject during the C10 infusion. There were no evident differences in motor patterns between nauseated and non-nauseated subjects during the C12 infusion.

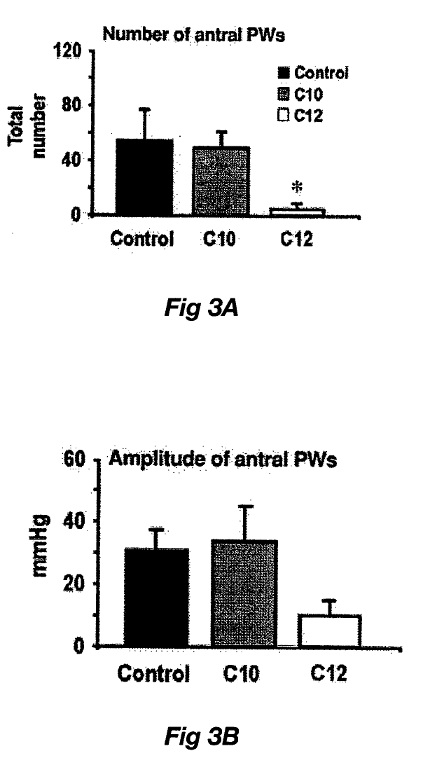

Antral pressures. There was a significant effect of treatment on the number of antral PWs (P=0.004; FIG. 3A). The infusion of C12 decreased the number of antral PWs compared with control (P=0.002) and C10 (P=0.003), whereas there was no difference between C10 and control (NS). There was a trend for the amplitude of antral pressures to differ between study conditions (treatment effect: P=0.080), and the amplitude tended to be lower during the C12 compared with both the C10 and control infusions (FIG. 3B).

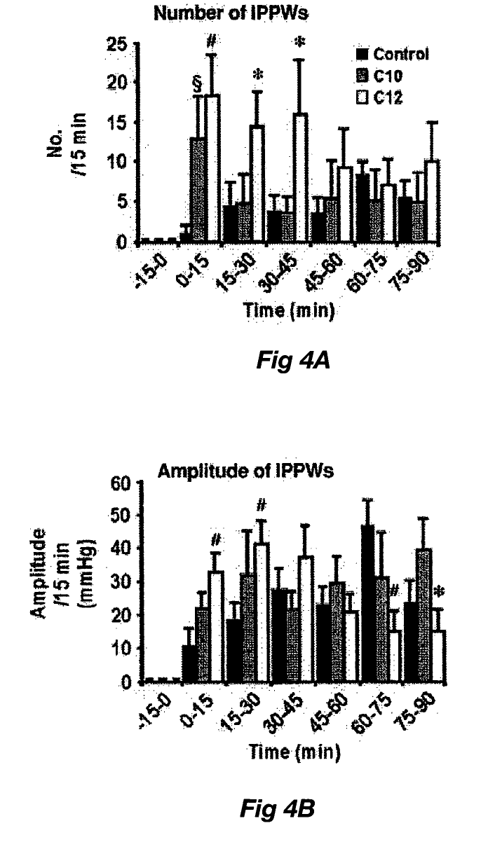

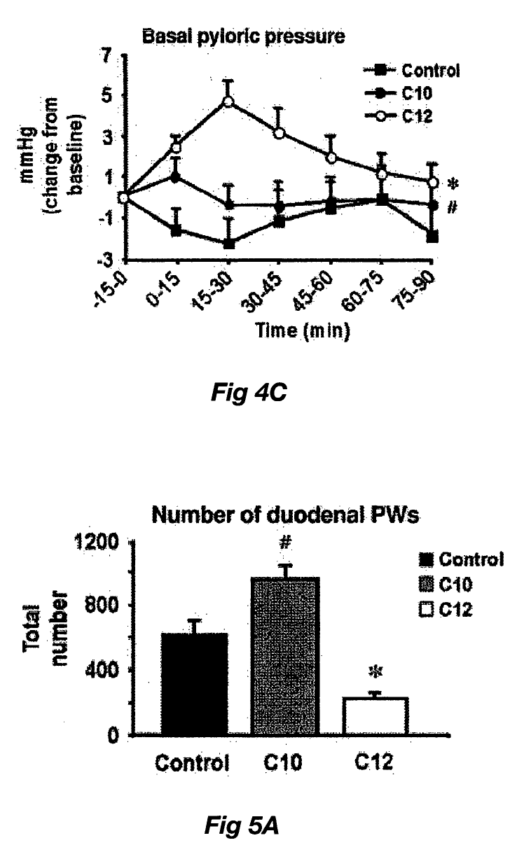

Pyloric pressures. PHASIC PRESSURES. A treatment X time interaction approached statistical significance for the number of IPPWs (P=0.062; FIG. 4A). The infusion of C12 increased the number of IPPWs between 15-30 and 30-45 min (P=0.046, P=0.015, respectively) compared with C10 and among 0-15, 15-30, and 30-45 min (P<0.001, P=0.019, and P=0.02, respectively) compared with control. C10 increased the number of IPPWs compared with control between 0 and 15 min (P=0.001).

There was a significant treatment X time interaction for the amplitude of IPPWs (P=0.008; FIG. 4B). The infusion of C12 increased the amplitude of IPPWs compared with control between 0-15 and 15-30 min (P=0.015 and P=0.011). C12 decreased the amplitude of IPPWs between 75 and 90 min compared with C10 (P=0.025) and between 60 and 75 min compared with control (P=0.010).

There were no significant differences between C10 and control throughout the infusion period (NS).

BASAL PRESSURE (“TONE”). There was a significant effect of treatment on basal pyloric pressure (P<0.001; FIG. 4C). The infusion of C12 increased basal pyloric pressure compared with both C10 (P=0.002) and control (P<0.001). There was also a significant difference between C10 and control (P=0.029).

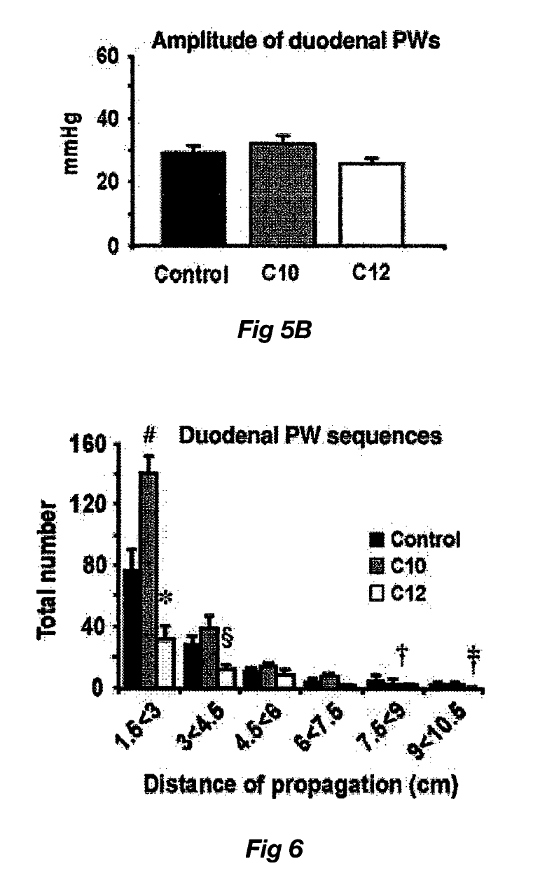

Duodenal pressures, PRESSURE WAVES. There was a significant effect of treatment on the number of duodenal PWs (P<0.001; FIG. 5A). The infusion of C12 decreased the number of duodenal PWs compared with both control (P=0.002) and C10 (P<0.001). In contrast, C10 increased the number of duodenal PWs compared with control (P=0.004). There were no differences in the amplitude of duodenal PWs between treatments (NS; FIG. 5B).

PRESSURE WAVE SEQUENCES. There was a significant effect of treatment on the number of duodenal PWSs travelling over two (i.e., 1.5 to <3 cm; P<0.001), three (i.e., 3 to <4.5 cm; P=0.023), six (i.e., 7.5 to <9 cm; P=0.007), and seven (i.e., 9 to <10.5 cm; P=0.021) channels (FIG. 6). The infusion of C12 decreased the number of waves that travelled over two channels compared with control (P=0.017) and C10 (P<0.001), whereas C10 increased the number of waves compared with control (P=0.002). The infusion of C12 decreased the number of waves that travelled over three channels compared with C10 (P=0.007), whereas there was no difference between C12 or C10 and control (NS). Both C12 (P=0.002) and C10 (P=0.043) decreased the number of waves that travelled over six channels compared with control. There was no difference between the C12 and C10 infusions (NS). C12 infusion also decreased the number of waves that travelled over nine channels compared with both control (P=0.021) and C10 (P=0.010). There was no difference between C10 and control (NS).

Plasma CCK and GLP-1 Concentrations

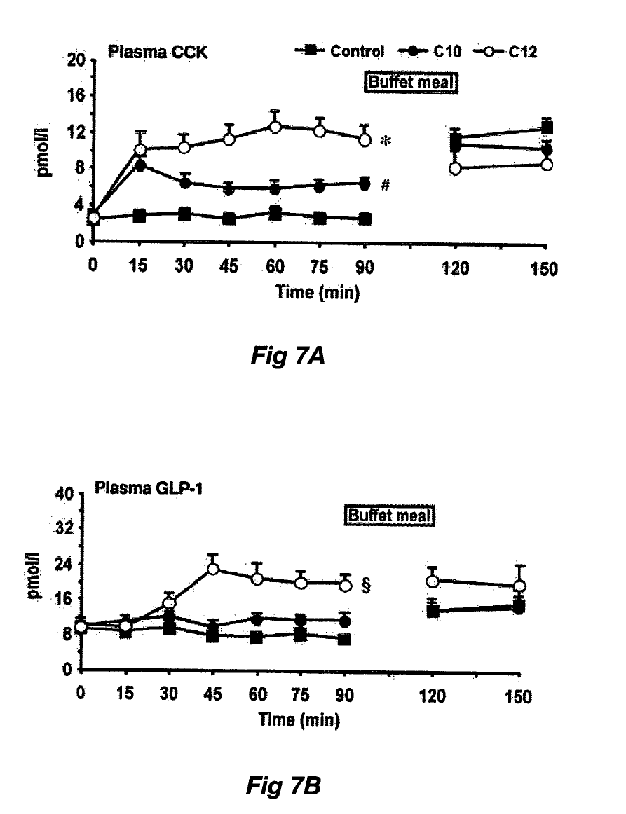

Baseline plasma CCK concentrations did not differ among study days. There was a significant effect of treatment on plasma CCK concentrations (P<0.001; FIG. 7A). The infusion of C12 increased plasma CCK compared with control (P <0.001) and C10 (P<0.001), where C12 produced a rapid rise in plasma CCK within 15 min, which then plateaued. However, C10 also increased plasma CCK concentrations, with a peak at 15 min, compared with control (P<0.001).

Baseline plasma GLP-1 concentrations did not differ among study days. There was a significant treatment X time interaction for plasma GLP-1 concentrations (P<0.001; FIG. 7B). The infusion of C12 increased plasma GLP-1 from 45 to 90 min compared with C10 (P<0.001) and from 30 to 90 min compared with control (P<0.05). There was no difference between C10 and control (NS).

During infusion of C12, the increases in plasma concentrations of CCK and GLP-1 were of similar magnitudes in nauseated and non-nauseated subjects.

After meal ingestion, plasma CCK concentrations increased during the control and C10 infusions, and at t=120 and 150 min there were no longer any differences between treatments (NS). In contrast, there was still a significant effect of treatment on plasma GLP-1 concentrations (P=0.013), which were higher after the C12 infusion compared with both control (P=0.007) and C10 (P=0.012), whereas there was no difference between C10 and control (NS).

Relationships Between the Desire to Eat and Nausea with Plasma Hormone Concentrations and Pyloric Pressures

Negative correlations were found between scores for the desire to eat and plasma CCK (r=−0.45, P<0.001), plasma GLP-1 (r=−0.38, P<0.001), and the sum of CCK and GLP-1 (r=−0.45, P<0.001). There was also a negative correlation between the desire to eat and basal pyloric pressure (r=−0.26, P=0.003). No significant correlations were found between the desire to eat and either the number or amplitude of IPPWs. There was a strong correlation between the score for the desire to eat at 90 min and subsequent energy intake (r=0.83, P<0.001).

There were correlations between scores for nausea and plasma CCK (r=0.52, P<0.001), plasma GLP-1 (r=0.45, P<0.001), and the sum of CCK and GLP-1 (r=0.52, P<0.001). Correlations were also evident between nausea and the number of IPPWs (r=0.32, P<0.001) and basal pyloric pressure (r=0.40, P<0.001). A negative correlation was observed between the score for nausea at 90 min and subsequent energy intake (r=−0.65, P=0.007).

### DISCUSSION

This experiment established that there are major differences in the effects of C12 and C10 fatty acids infused into the duodenum at a dose of 0.375 kcal/min on appetite, APD motility, and gastrointestinal hormones in healthy males.

Compared with an isocaloric dose of C10 and control, C12 reduced appetite and subsequent energy intake at a buffet meal, increased basal pyloric pressure and IPPWs, decreased antral and duodenal pressure waves, shortened the length of propagation of duodenal PWs, and increased plasma CCK and GLP-1 concentrations. Whereas C10 stimulated CCK release, albeit less than C12, no effect on GLP-1 was evident.

A striking observation was that the small amount of C12 (total amount of energy delivered: −141 kJ) markedly attenuated appetite-related sensations and subsequent energy intake; C12 reduced energy intake by ˜2,857 kJ compared with the control infusion. In a previous study in humans, intraduodenal infusion of C18:1 for 90 min (providing ˜288 kJ) reduced energy intake by ˜1,580 kJ.

Accordingly, intraduodenal infusion of C12 is considered to be a more potent suppressant of energy intake in humans than isoenergetic amounts of C18:1, as is the case in rats. However, the unexpected occurrence of nausea in our experiments (discussed below) during the infusion of C12 may affect this interpretation. In our previous studies in which triglyceride emulsions were infused directly into the duodenum at a rate of ˜2.8 kcal/min [corresponding to observed rates of gastric emptying], the gastrointestinal responses (including stimulation of pyloric motility and plasma CCK) were maximal within 30-45 min of the start of the infusions. However, in the present experiment, similar effects were evident during C12 infusion at a much lower dose (0.375 kcal/min) with an onset at ˜15 min (i.e., 15-30 min earlier than that observed during previous triglyceride infusions, suggesting that only very small amounts of fatty acid are required to bring about gastrointestinal effects). It is possible that the gastrointestinal tract is more sensitive to the action of lauric acid, as opposed to longer-chain fatty acids.

Although lauric acid is contained in a variety of foods, including coconut milk (up to 50%), butter (5-8%) and beef, it, at least in most cases, does not represent a major component of our total energy intake: on the basis of compositions of stored bodily fatty acids, it is estimated that C12 represents ˜6% of dietary fatty acids. Breast milk, which contains up to 20% of lauric acid, is only part of the human diet in early life.

Previous studies have determined that fatty acids with ≧12 carbon atoms slow gastric emptying, induce proximal gastric relaxation, and reduce antral PWs to a greater extent than fatty acids with <12 carbon atoms, but this is the first study to evaluate effects on pyloric and duodenal pressures. The intraduodenal infusion of C12 caused a substantial increase in basal pyloric pressure and the number and amplitude of IPPWs and decreased the number of antral and duodenal PWs as well as the number of PWSs that travelled over two, three, six, and seven channels. The motor patterns observed during C12 infusion are known to be associated with a slowing of gastric emptying, which is thought to play a role in reducing energy intake. C10 also stimulated IPPWs; however, this effect was confined to the first 15 min of the infusion and subsequently disappeared. In contrast to C12, C10 increased the number of PWSs that travelled over two channels. These differences in motor patterns induced by C10 and C12, particularly the effects on basal pyloric pressure and IPPWs, may underlie their differential rates of gastric emptying.

It has been suggested that fatty acids with a chain length of ≧12 carbon atoms increase plasma CCK concentrations, whereas fatty acids with a chain length of ≦11 carbon atoms do not. For example, in humans, duodenal infusion of C18, but not C8, and gastric infusion of C12, but neither C11 nor C10, were reported to stimulate CCK secretion. In the latter study, the authors concluded that C10 had no effect because C10 was associated with a rise in plasma CCK concentration that did not differ from that of the control solution (Tween 80). In contrast, our data establish that C10, when infused as a sodium salt without the confounding effects of a dispersing agent, stimulated plasma CCK (although the effect of C10 was substantially less than that of C12), consistent with earlier studies in both animals and humans. The magnitude of the rise in plasma CCK during the C12 infusion (˜12 pmol/l in the first 15 min) also demonstrates the greater potency of free fatty acids compared with duodenal triglyceride infusion, which, at doses of ˜2.8 kcal/min, results in plasma concentrations of ˜6 pmol/l. This study represents the first instance of the evaluation of the effects of fatty acids of different chain lengths on GLP-1 secretion; in contrast to CCK, C12 but not C10 increased plasma GLP-1 concentrations. It is noteworthy that CCK was released almost immediately after the start of the C10 and C12 infusions, whereas there was a 30-min delay until plasma GLP-1 concentrations rose, indicating a discordance between CCK and GLP-1 release, which may relate to the release of these peptides by localized contact with fatty acids at a more distal, ileal, site for GLP-1 as opposed to a more proximal, jejunal, site for CCK.

In humans, intravenous infusions of both CCK and GLP-1 inhibit energy intake and modulate gastrointestinal motility, consistent with the effects produced by C12. Our present observations do not establish that CCK and/or GLP-1 mediate the effects on energy intake and motility (and it still should be recognized that plasma hormone concentrations probably do not precisely reflect events at the sites of release) but favour the concept that the effects of C12 may reflect an interaction between CCK and GLP-1 and possibly other gut peptides. That the two peptides acted together on appetite and other parameters is strongly suggested by the time courses of some of our observations (FIGS. 1 and 6). For example, after C12, the desire to eat decreased progressively over the entire 90 min of infusion, whereas nausea increased progressively; however, plasma CCK levels were nearly constant after 15 min, whereas the later rise in GLP-1 may have added to the CCK signal to produce the observed effects. In contrast, our observations during the C10 infusion suggest that the magnitude of CCK secretion in the absence of GLP-1 was insufficient to sustain the effect on motility or induce changes in appetite perceptions and energy intake. Possible interactions between CCK and GLP-1, therefore, deserve further evaluation; recent studies have emphasized the importance of the interaction between signals arising from the gut, e.g., gastric distension with duodenal nutrients or intravenous CCK.

In our experiments, five of eight subjects experienced nausea during the C12 infusion. This might potentially have resulted from a transitory cytotoxic effect of fatty acids. Cytotoxicity increases with fatty acid concentration, chain length, and degree of unsaturation, so that C12 is half as cytotoxic as C18:1 at similar concentrations, and the cytotoxicities of isomolar concentrations of C10 and C12 are similar. Because Matzinger et al. (6) infused 280 mM C18:1 (i.e., some 2.6 times the concentration we used) intraduodenally without observing nausea, it is unlikely that the 106 mM C12 infused in our study produced nausea as a result of cytotoxicity. Furthermore, the concentration of C10 was slightly greater than that of C12 (123 vs. 106 mM). The differential effects of C12 and C10 on the secretion of GLP-1 and CCK may be important. Intravenous administration of both GLP-1 and CCK, albeit in pharmacological doses, can induce nausea. Our dose of C12 resulted in high levels of both GLP-1 (40% higher than after the ingestion of ˜4,528 kJ from the buffet meal after the control infusion) and CCK (comparable to postprandial levels after the control infusion), which may support this concept. Although we observed correlations between CCK and GLP-1 with nausea, it is important to recognize that correlations cannot be taken to infer any cause-effect relationships. The absence of an association between the perception of “sickness” and plasma CCK has recently been reported (7). Alternatively, the pronounced nausea during C12, but not C18:1, may reflect the unique effect of C12 on pancreatic bicarbonate secretion. Luminal C12 differs from C10, C16, C18:0, C18:1, and C18:2 by stimulating more than twice as much secretion of pancreatic bicarbonate as these other fatty acids, a vasoactive intestinal peptide-, secretin-or neurotensin-like effect.

It should be recognized that the several effects of C12 on gastroduodenal motility that differed from those evoked by C10 are not characteristic of nausea. It is well established from animal studies that vomiting (presumably preceded by nausea) inhibits gastric contractions and produces retrograde duodenal peristalsis. In contrast, C12 did not abolish but did shorten the length of antegrade peristalsis in the duodenum. Increases in pyloric tone and IPPWs stimulated by C12 (vs. C10) have not been described during nausea but are typical of the physiological effects of intraduodenal fat. Furthermore, subjects who experienced no nausea exhibited similar pressure patterns. Thus it is unlikely that nausea accounted for the differing pressure patterns during C12 vs. C10. In addition, although it is clear that C12-induced nausea substantially contributed to inhibition of energy intake, and our data indicate a correlation between nausea at 90 min and energy intake, C12 also inhibited energy intake in the absence of nausea.

Although the mechanisms underlying the discrepant effects of C10 and C12 on appetite and gastrointestinal function are incompletely understood, differences in the postabsorptive processing of the two fatty acids are likely to be important. In rats, fatty acids with chain lengths of ≦11 carbon atoms are transported from the absorptive cell directly into the systemic circulation via the portal vein (5), whereas fatty acids with chain lengths of ≧12 carbon atoms are absorbed into the lymphatic circulation as chylomicrons. When chylomicron transport was blocked in rats through the use of luminal L-pluronic acid, fat-induced suppression of energy intake, inhibition of gastric emptying and release of CCK, and stimulation of vagal discharge by intraluminal oleic acid were blocked. Moreover, results from a recent study in rats suggest that chylomicrons, or chylomicron derivatives, activate CCK-A receptors on vagal afferent nerve fibers by releasing CCK. The close relationship between the transport of lymphatic chylomicrons and signaling pathways evident in these experiments may, therefore, explain why C12 has a greater effect on appetite, APD PWs, and plasma hormone concentrations than C10. However, it must be recognized that a small percentage of fatty acids with ≧12 carbon atoms are transported via the portal vein, whereas a small percentage of fatty acids with ≦10 carbon atoms are transported through the lymphatic circulation, potentially explaining some of the effects of C10 observed in our study.

This study demonstrates the marked differences in the effects of isoenergetic doses of C12 and C10 administered intraduodenally. C12, but not C10, inhibited appetite and energy intake and increased plasma GLP-1 concentrations, and C12 modulated APD motility patterns and increased plasma CCK concentrations to a greater extent than C10. Thus the use of C12, released into the duodenum, as a novel treatment for the reduction in overall energy intake while maintaining the satiety effect has been shown.

A further aspect of the present invention has been to prepare compositions containing 90% of C12 formulated for oral delivery, to provide a solid or liquid dispersed form that ensures delivery of the C12 into the stomach. This then ensures that there is immediate and constant transfer from the stomach into the duodenum.

This is achieved by formulating the composition to include other forms of dodecanoic acid including the addition of either lauric acid, lauric acid glycerides, their derivatives or a combination of the lauric acid glyceride derivatives so as to provide the effect of providing a liquid or solid dispersion in the stomach.

Moreover, the use of glyceryl dodecanoate, glyceryl 1,3-didodecanoate and glyceryl tridodecanoate and derivatives and mixtures thereof require digestion prior to eliciting an appetite reducing effect, which occurs by release of dodecanoic acid/dodecanoate. Accordingly, when using any of the glyceryl derivatives of dodecanoic acid the appetite suppression effect is more moderate but the time period over which the subject feels the effect may be prolonged. The rate at which the glycerol derivatives of dodecanoic acid are digested is however faster than that of the longer chain glycerol derivatives such as triolein (C18).

The satiety agent can be administered in conventional oral compositions, including tablets, coated tablets, hard and soft gelatin capsules, emulsions or suspensions.

In preference, pharmaceutically accepted delivery agents which can be used for tablets, coated tablets, dragees and hard gelatin capsules are lactose, other sugars and sugar alcohols like sorbitol, mannitol, maltodextrin, micorcrystalline cellulose, sodium chloride or other fillers; gelatin, polyvinylpyrrolidone, starch, Acacia or other binders, stearic acid or its salts, talc, polyethyle glycol, liquid paraffin, sodium lauryl sulfate, colloidal silica or other glidants and lubricants, other surfactants like, Brij 96, or Tween 80, polyoxyethylene-polypropylene copolymers, eg Lutrol; disintegrants like sodium starch glycolate, cellulose or derivatives thereof; polymers such as povidone, crospovidone.

Suitable carriers for soft gelatin capsules are, for example, vegetable oils, waxes, fats, semi-solid and liquid polyols and the like. Additionally, the pharmaceutical compositions can contain preserving agents, solubilisers, stabilising agents, wetting agents, emulsifying agents, sweetening agents, colouring agents, flavouring agents, salts for varying the osmotic pressure, buffers, coating agents and antioxidants. They can also contain still other therapeutically valuable substances. The formulations may conveniently be presented in unit dosage form and may be prepared by any methods known in those skilled in the art of pharmaceutical preparation.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent compositions and methods.

### REFERENCES

1. Feinle C, O'Donovan D G, Doran S, Andrews J M, Wishart J, Chapman i, and Horowitz M. Effects of fat digestion on appetite, APD motility, and gut hormones in response to duodenal fat infusion in humans. Am J Physiol 284: G798-807, 2003.
2. Stunkard A J and Messick S. The three-factor eating questionnaire to measure dietary restraint, disinhibition and hunger. J Psychosom Res 29: 71-83, 1985.
3. Macintosh C G, Andrews J M, Jones K L, Wishart J M, Morris H A, Jansen J B, Morley J E, Horowitz M, and Chapman I M. Effects of age on concentrations of plasma cholecystokinin, glucagon-like peptide-1, and peptide YY and their relation to appetite and pyloric motility. Am J Clin Nutr 69: 999-1006, 1999.
4. Orskov C, Jeppesen J, Madsbad S, and Hoist J J. Proglucagon products in plasma of noninsulin-dependent diabetics and nondiabetic controls in the fasting state and after oral glucose and intravenous arginine. J Clin Invest 87: 415-423, 1991.
5. Jones K L, Horowitz M, Carney B I, Sun W M, and Chatterton B E. Effects of cisapride on gastric emptying of oil and aqueous meal components, hunger, and fullness. Gut 38: 310-315, 1996.
6. Matzinger D, Degen L, Drewe J, Meuli J, Duebendorfer R, Ruckstuhl N, D'Amato M, Rovati L, and Beglinger C. The role of long chain fatty acids in regulating food intake and cholecystokinin release in humans. Gut 46: 688-693, 2000.
7. Nolan L J, Guss J L, Liddle R A, Pi-Sunyer F X, and Kissileff H R. Elevated plasma cholecystokinin and appetitive ratings after consumption of a liquid meal in humans. Nutrition 19: 553-557, 2003.

1. A satiety modifying composition comprising a pharmaceutically acceptable satiety agent selected from a group consisting of dodecanoic acid, glyceryl dodecanoate, glyceryl 1,3-didodecanoate, glyceryl tridodecanoate and derivatives and mixtures thereof, and a pharmaceutically acceptable delivery agent.

2. A composition according to claim 1, wherein the composition is formulated for predominant release of the satiety agent in the stomach.

3. A composition according to claim 2, wherein the concentration of satiety agent is at least 80%.

4. A composition according to claim 3, wherein the concentration of satiety agent is at least 90%.

5. A composition according to claim 4, wherein the satiety agent is present in the amount ranging from 0.5 grams to 15 grams.

6. A composition according to claim 5, wherein satiety agent is present in the amount ranging from 2 grams to 10 grams.

7. A composition according to claim 6, wherein satiety agent is present in the amount ranging from 3 grams to 6 grams.

8. A composition according to claim 7, wherein the satiety agent is formulated to be dispersed in the stomach between 15° C. and 40° C.

9. A composition according to claim 8 wherein the satiety agent is formulated to be dispersed in the stomach between 18° C. and 25° C.

10. A composition according to claim 9, wherein the satiety agent is in liquid form.

11. An appetite control composition comprising a satiety agent selected from the group consisting of dodecanoic acid, glyceryl dodecanoate, glyceryl 1,3-didodecanoate and glyceryl tridodecanoate and derivatives and mixtures thereof, and a delivery agent designed to release the satiety agent predominantly in the stomach.

12. A method of controlling appetite comprising the steps of administering to a subject whose appetite is to be controlled a composition comprising a satiety agent selected from the group consisting of dodecanoic acid, glyceryl dodecanoate, glyceryl 1,3-didodecanoate and glyceryl tridodecanoate and derivatives and mixtures thereof, and a delivery agent designed to release the satiety agent predominantly in the stomach.

13. The method of claim 12, wherein the composition is ingested 5-60 min prior to the ingestion of food.

14. The method of manufacture of a medicament for the treatment of obesity, composition comprising a satiety agent selected from the group consisting of dodecanoic acid, glyceryl dodecanoate, glyceryl 1,3-didodecanoate and glyceryl tridodecanoate and derivatives and mixtures thereof.

15. A method of increasing a feeling of satiety in a patient which comprises a step of effecting by oral ingestion a composition comprising a pharmaceutically acceptable satiety agent selected from a group consisting of any one alone or any two or more in combination the group being dodecanoic acid, glyceryl dodecanoate, glyceryl 1,3-didodecanoate and glyceryl tridodecanoate and derivatives and mixtures thereof, and a pharmaceutically acceptable delivery agent.

16. A method as claimed in claim 15 in which the pharmaceutically acceptable delivery agent is either a capsule within which the active agent is held or a coating either coating as a whole or smaller portions the active agent or agents where the capsule or coating is such as to resist release of the active agent when orally consumed until within the stomach of a patient and then allow release of the active agent while within a major extent within the stomach of a patient.

17. A method as in claim 15 wherein the step is effected so that the ingestion is effected at least after 5 minutes and not longer than 60 minutes before commencement of expected oral partaking of food by a user patient.

18. A method as in claim 15 wherein the step is effected so that the ingestion is effected at least 5 minutes and not longer than 60 minutes before the commencement of oral ingestion of food by a patient.
